(19) United States
(12) Patent Application Publication
(10) Pub. No.: US 2012/0003303 A1
(43) Pub. Date: Jan. 5, 2012

(54) ORAL ENTERIC ANTIDEPRESSANT FORMULATION

(75) Inventors: Barry Scott Brand, Cary, NC (US); James Cecil Free, Apex, NC (US); Daniel Joseph Burch, Durham, NC (US); Mark Joseph Baric, Cary, NC (US)

(73) Assignee: CeNeRx Biopharma, Inc, Cary, NC (US)

(21) Appl. No.: 13/143,408

(22) PCT Filed: Jan. 8, 2010
(86) PCT No.: PCT/US10/20459

§ 371 Date: Sep. 21, 2011

Related U.S. Application Data
(60) Provisional application No. 61/143,764, filed on Jan. 9, 2009.

(51) Int. Cl. A61K 31/39; A61P 9/12; A61K 9/14; C07D 327/08; A61K 9/48

(52) U.S. Cl. 424/451; 549/16; 424/490; 424/494; 514/434

(57) ABSTRACT

Pharmaceutical presentations of phenoxathiin-based MAO-A inhibitors are disclosed whereby the MAO receptors are protected from binding to active ingredient in the stomach. Particular phenoxathiin-based MAO-A inhibitors include those of the following formula: (I) wherein n is 0, 1 or 2; R1 is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl. A wide variety of enteric mechanisms may be utilized so as to provide release of the active ingredient essentially out of the environment of the stomach after ingestion as a pharmaceutical presentation, such as a tablet or capsule. Presentations include enteric coated tablets, enteric coated capsules, capsules containing enteric coated beads.

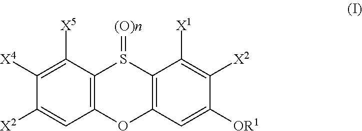

### BACKGROUND

1. Technical Field

Provided herein are oral enteric pharmaceutical formulations, products and related methods. In particular, provided herein are oral enteric pharmaceutical formulations, products comprising 3-fluoro-7-(2,2,2-trifluoroethoxy)phenoxathiin 10,10-dioxide as an active ingredient, and related methods.

2. Background

The transient elevation of blood pressure, leading in some cases to hypertensive crisis, has been noted in patients treated with monoamine oxidase inhibitor (MAOI) agents, such as phenelzine, isocarboxazide, ipraniazid, and tranylcypromine following the consumption of tyramine-rich dietary foods and beverages. This acute form of hypertension, similar to that seen in patients with pheochromocytoma, has been referred to in the medical literature as the “cheese effect” or “cheese reaction” because of the high tyramine content found in some aged cheeses. Because of this potentially dangerous food reaction, physicians have been reluctant to prescribe MAOIs even though they are highly effective in the treatment of major depressive disorder, social phobia and panic attack.

Therefore, there remains a need for suitable MAOIs that do not elicit dangerous food reactions or require strict dietary restrictions. The formulations, products and methods provided herein address this need and provide additional advantages.

### SUMMARY

Enteric pharmaceutical presentations of phenoxathiin-based MAO-A inhibitors are disclosed whereby the MAO receptors are protected from binding to active ingredient in the stomach. Particular phenoxathiin-based MAO-A inhibitors include those of the following formula:

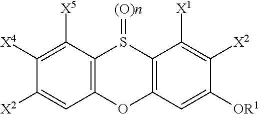

wherein n is 0, 1 or 2; R¹ is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl. Examples of phenoxathiin-based MAO-A inhibitors include, but are not limited to, 3-fluoro-7-(2,2,2-trifluoroethoxy)phenoxathiin 10,10-dioxide (hereinafter “CX157”) of the following formula:

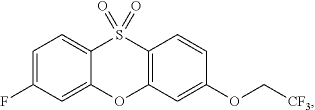

3-(2,2,2-trifluoroethoxy)phenoxathiin 10,10-dioxide (hereinafter “CX009”) of the following formula:

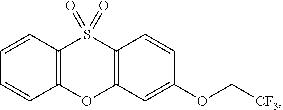

and 3-(2,2,2-trifluoro-1-methylethoxy)phenoxathiin 10,10-dioxide (hereinafter “CX2614”) of the following formula:

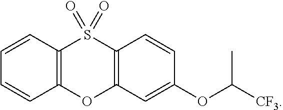

Such presentations do not elicit dangerous food reactions or require strict dietary restrictions by virtue of permitting absorption of the MAO-A inhibitor at a portion of the digestive tract which does not prevent MAO receptors from binding dietary tyramine.

In some embodiments, such presentations are in tablet form, capsule form or a core sheathed in an annular body. In some embodiments, such presentations comprise an enteric coating which is essentially not dissolvable in the stomach surrounding a core which comprises said active ingredient. In some embodiments, such presentations contain 3-fluoro-7-(2,2,2-trifluoroethoxy)phenoxathiin 10,10-dioxide as the sole active ingredient. In some embodiments, 3-fluoro-7-(2,2,2-trifluoroethoxy)phenoxathiin 10,10-dioxide is characterized as having a melting point at about 169-175° C. In some embodiments, 3-fluoro-7-(2,2,2-trifluoroethoxy)phenoxathiin 10,10-dioxide is characterized as being in crystalline form and having an x-ray powder diffraction peak at 2θ=11.0°, using CuK_{α} radiation.

In some embodiments, such presentations comprise: (a) a core consisting of 3-fluoro-7-(2,2,2-trifluoroethoxy)phenoxathiin 10,10-dioxide and one or more pharmaceutical excipients; (b) an optional separating layer; (c) an enteric layer comprising hydroxypropylmethylcellulose acetate succinate (HPMCAS) and a pharmaceutically acceptable excipient; and (d) an optional finishing layer. In some such embodiments the separating layer (b) is present. In some such embodiments, the core comprises an inert bead on which the 3-fluoro-7-(2,2,2-trifluoroethoxy)phenoxathiin 10,10-dioxide is deposited as a layer comprising said one or more pharmaceutical excipients.

In some embodiments, such presentations are tablets containing about 50 to 500 milligrams of 3-fluoro-7-(2,2,2-trifluoroethoxy)phenoxathiin 10,10-dioxide. In some embodiments, such presentations comprise 3-fluoro-7-(2,2,2-trifluoroethoxy)phenoxathiin 10,10-dioxide and are adapted to retard or inhibit the release of 3-fluoro-7-(2,2,2-trifluoroethoxy)phenoxathiin 10,10-dioxide in the stomach. In some such embodiments, the presentation is a tablet, a capsule, or a core sheathed in an annular body. In some such embodiments, the presentation is a tablet. In some such embodiments, the presentation comprises an enteric coating.

### BRIEF DESCRIPTION OF THE DRAWINGS

FIG. 1 is a diagram of the enzymatic barriers and enzymes involved in the biotransformation of orally administered tyramine in an unmedicated subject (upper portion) and in a MAO-A inhibited subject (lower portion). In humans, the activity of MAO-A and MAO-B is as follows: intestinal mucosa, 90% and 10%: liver, 30% and 70%: adrenergic nerve terminal, 100% and 0%, respectively. Abbreviations: HPAA=p-hydroxyphenylacetic acid; tyramine=free tyramine; Ty-SO4=tyramine sulfate; NA=noradrenaline; Oct.=octopamine; COMT=catachol-O-methyltransferase.

### DETAILED DESCRIPTION

Monoamine oxidase inhibitor (MAOI) agents can cause dangerous food reactions following the consumption of tyramine-rich dietary foods and beverages. This dangerous side-effect has minimized the use of MAOIs even though they are highly effective in the treatment of major depressive disorder, social phobia and panic attack. Reversible inhibitors of monoamine oxidase type-A (RIMAs) are a family of psychiatric drugs and natural compounds that inhibit monoamine oxidase temporarily and reversibly. The pharmacological properties of the RIMAs permit oral administration in antidepressant doses of these agents while lessening the risk of the cheese reaction. However, therapeutic doses of some RIMAs can still potentiate the tyramine pressor effect as much as 40- to 50-fold. As a result, RIMAs also are seldom used therapeutically.

As provided herein, greater safety factors for a particular class of RIMAs, termed phenoxathiin-based MAO-A inhibitors (also referred to herein as “active” or “active ingredient”), as defined above in the Summary, can be achieved through an enteric formulation that release in the intestine so as to avoid competing with dietary tyramine for MAO-A in the gastrointestinal and hepatic tissues. Such a formulation is particularly effective with CX157 since this RIMA is devoid of inhibitory actions on MAO-B, thus allowing tyramine inactivation through the MAO-B pathway. Thus the specific and reversible properties of CX157 as a MAO-A inhibitor provide a favorable profile for a weak potentiating effect on the oral tyramine pressor effect.

Provided herein are formulations engineered to initiate drug release in the middle to lower portions of the small intestine, with a delayed release time of greater than, for example, approximately 1 hour, 1.25 hours, 1.5 hours, 1.75 hours or 2 hours after dosing. Such pharmaceutical formulations are manufactured in such a way that the product passes unchanged through the stomach of the patient, and dissolves and releases the active ingredient when it leaves the stomach and enters the middle and lower portions of the small intestine. Such formulations can be in tablet or pellet form, where the active ingredient is in the inner part of the tablet or pellet and is enclosed in a film or envelope, the “enteric coating,” which is insoluble in acid environments, such as the stomach, but is soluble in near-neutral environments such as the small intestine. The instant enteric coating-containing formulations avoid much of the drug competition with dietary tyramine for MAO-A since dietary tyramine is rapidly absorbed and metabolized in the stomach and upper portion of the small intestine and the liver with an average Tmax of 1.25 hours. In this regard, human plasma pharmacokinetic data of tyramine, administered with food in a capsule, in an oral dose of 200 mg demonstrated a rapid absorption of tyramine with a Tmax achieved within 1.25 hours and non-detectable levels observed 3-4 hrs after dosing.

In accordance with the above, various formulations and presentations of phenoxathiin-based MAO-A inhibitors, and particularly, of CX157 are provided herein. For example, clinical trial and commercial tablets of a phenoxathiin-based MAO-A inhibitor such as CX157 can be coated, encapsulated or otherwise treated so as to render the tablet enteric.

As used herein, all expressions of percentage, ratio, proportion and the like, will be in weight units unless otherwise stated. Expressions of proportions of the enteric product will refer to the product in dried form, after the removal of the water in which many of the ingredients are dissolved or dispersed. The term “sugar” refers to a sugar other than a reducing sugar. A reducing sugar is a carbohydrate that reduces Fehling's (or Benedict's) or Tollens' reagent. All monosaccharides are reducing sugars as are most disaccharides with the exception of sucrose. One common binding or filling agent is lactose. This excipient is particularly useful for tablets since it compresses well, is both a diluent and binder, and is cheap. However, it is a reducing sugar and it may be that the active ingredient interacts with lactose both at room temperature and under accelerated stability conditions (heat). Therefore, avoidance of lactose and other reducing sugars from formulations comprising the active ingredient may be important. As discussed below, sucrose is a particular sugar.

In a particular enteric product, a core of active is surrounded by an enteric coat and formed into a pellet. The pellets can then be loaded into gelatin capsules. The various components and layers of the pellet will be individually discussed as follows, together with the methods of adding the different ingredients to build up the pellet.

### A. The Core

A particular core for the pellet is typically prepared by applying an active ingredient-containing layer to an inert core. Such inert cores are conventionally used in pharmaceutical science, and are readily available. A particular core is one prepared from starch and sucrose, for use in confectionery as well as in pharmaceutical manufacturing. However, cores of any pharmaceutically acceptable excipient can be used, including, for example, microcrystalline cellulose, vegetable gums, waxes, and the like. The primary characteristic of the inert core is to be inert, with regard both to the active ingredient and the other excipients in the pellet and with regard to the subject who will ultimately ingest the pellet.

The size of the cores depends on the desired size of the pellet to be manufactured. In general, pellets can be as small as 0.1 mm, or as large as 2 mm. Particular cores are from about 0.3 to about 0.8 mm, in order to provide finished pellets in the size range of from about 0.5 to about 1.5 mm in diameter. For instance, the cores can be of a reasonably narrow particle size distribution, in order to improve the uniformity of the various coatings to be added and the homogeneity of the final product. For example, the cores can be specified as being of particle size ranges such as from 18 to 20 U.S. mesh, from 20 to 25 U.S. mesh, from 25 to 30 U.S. mesh, or from 30 to 35 U.S. mesh to obtain acceptable size distributions of various absolute sizes.

The amount of cores to be used can vary according to the weights and thicknesses of the added layers. In general, the cores comprise from about 10 to about 70 percent of the product. More particularly, the charge of cores represents from about 15 to about 45 percent of the product.

When manufacture of the pellet begins with inert cores, the active ingredient can be coated on the cores to yield a final drug concentration of about 10 to about 25 percent of the product, in general. The amount of active ingredient depends on the desired dose of the drug and the quantity of pellets to be administered. The dose of active ingredient is in the range of about 50-500 mg, more particularly about 60-200 mg, and the usual amount of pellets is that amount which is conveniently held in gelatin capsules. The volume of gelatin capsules can range of from about 15% to about 25% of active in the present product.

A convenient manner of coating the cores with active ingredient is the “powder coating” process where the cores are moistened with a sticky liquid or binder, active ingredient is added as a powder, and the mixture is dried. Such a process is regularly carried out in the practice of industrial pharmacy, and suitable equipment is known in the art.

Such equipment can be used in several steps of the present process. This process can be conducted in conventional coating pans similar to those employed in sugar coating processes. This process can be used to prepare pellets.

Alternately, the present product can be made in fluidized bed equipment (using a rotary processor), or in rotating plate equipment such as the Freund CF-Granulator (Vector Corporation, Marion, Iowa). The rotating plate equipment typically consists of a cylinder, the bottom of which is a rotatable plate. Motion of the mass of particles to be coated is provided by friction of the mass between the stationary wall of the cylinder and the rotating bottom. Warm air can be applied to dry the mass, and liquids can be sprayed on the mass and balanced against the drying rate as in the fluidized bed case.

In some embodiments, a powder coating is applied. In such embodiments, the mass of pellets can be maintained in a sticky state, and the powder to be adhered to them, active ingredient in this case, can be added continuously or periodically and adhered to the sticky pellets. When all of such active has been applied, the spray can be stopped and the mass allowed to dry in the air stream. It can be appropriate or convenient to add some inert powders to the active ingredient.

Additional solids can be added to the layer with active ingredient. These solids can be added to facilitate the coating process as needed to aid flow, reduce static charge, aid bulk buildup and form a smooth surface. Inert substances such as talc, kaolin, and titanium dioxide, lubricants such as magnesium stearate, finely divided silicon dioxide, crospovidone, and non-reducing sugars, e.g., sucrose, can be used. The amounts of such substances are in the range from about a few tenths of 1% of the product up to about 20% of the product. Such solids are typically of fine particle size, e.g., less than 50 micrometers, to produce a smooth surface.

The active ingredient can be made to adhere to the cores by spraying a pharmaceutical excipient which is sticky and adherent when it is wet, and dries to a strong, coherent film. Those skilled in the art are aware of and conventionally use many such substances, most of them polymers. Particular such polymers include hydroxypropylmethylcellulose, hydroxypropylcellulose and polyvinylpyrrolidone. Additional such substances include methylcellulose, carboxymethylcellulose, acacia and gelatin, for example. The amount of the adhering excipient can be in the range from about 4% to about 12% of the product, and depends in large part on the amount of active to be adhered to the core.

The active ingredient can also be built up on the cores by spraying a slurry comprising active suspended in a solution of the excipients of the active layer, dissolved or suspended in sufficient water to make the slurry sprayable. Such a slurry can be milled through a machine adapted for grinding suspension in order to reduce the particle size of active. Grinding in suspension form can be desirable because it avoids dust generation and containment problems which arise in grinding dry powder drugs. A particular method for applying this suspension is the pharmaceutical fluidized bed coating device, such as the Wurster column, which consists of a vertical cylinder with an air-permeable bottom and an upward spraying nozzle close above the bottom, or a downward-spraying nozzle mounted above the product mass. The cylinder is charged with particles to be coated, a sufficient volume of air is drawn through the bottom of the cylinder to suspend the mass of particles, and the liquid to be applied is sprayed onto the mass. The temperature of the fluidizing air is balanced against the spray rate to maintain the mass of pellets or tablets at the desired level of moisture and stickiness while the coating is built up.

On the other hand, the core can comprise a monolithic particle in which the active ingredient is incorporated. Such cores can be prepared by the granulation techniques which are wide spread in pharmaceutical science, particularly in the preparation of granular material for compressed tablets. The cores can be prepared by mixing the active into a mass of pharmaceutical excipients, moistening the mass with water or a solvent, drying, and breaking the mass into sized particles in the same size range as described above for the inert cores. This can be accomplished via the process of extrusion and marumerization.

The core for the pellet can also be prepared by mixing active with conventional pharmaceutical ingredients to obtain the desired concentration and forming the mixture into cores of the desired size by conventional procedures, including but not limited to the process of R. E. Sparks et al., U.S. Pat. Nos. 5,019,302 and 5,100,592, incorporated by reference herein.

A particular protected core of the enteric pharmaceutical product comprises 3-fluoro-7-(2,2,2-trifluoroethoxy)phenoxathiin 10,10-dioxide (also referred to herein as CX157) of the following formula:

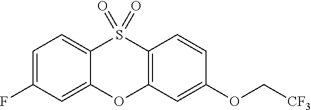

as an active ingredient. Another particular protected core of the enteric pharmaceutical product comprises 3-(2,2,2-trifluoroethoxy)phenoxathiin 10,10-dioxide (also referred to herein as CX009) of the following formula:

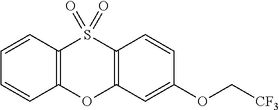

as an active ingredient. Another particular protected core of the enteric pharmaceutical product comprises 3-(2,2,2-trifluoro-1-methylethoxy)phenoxathiin 10,10-dioxide (hereinafter “CX2614”) of the following formula:

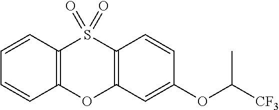

as an active ingredient. Methods for preparation of the above phenoxathiin-based MAO-A inhibitors and other phenoxathiin-based MAO-A inhibitors are known in the art, as exemplified in U.S. Pat. No. 6,110,961, which is incorporated by reference herein in its entirety.

Also provided herein are oral compositions such as tablets or capsules containing said active ingredient which have a low excipient load such that once or twice a day dosing is possible, preferably with one or two such compositions being administered at each dosing. The enteric product provided herein can utilize any physical form of the active ingredient. When the active pharmaceutical ingredient is CX157, the active ingredient can be in the “high melt” crystalline form.

The “high melt” crystalline form for CX157 is taught in U.S. application Ser. No. 11/773,892, which is incorporated by reference herein in its entirety, where “Form A” of the aforementioned application is the form referred to herein as “high melt.” Briefly, the high melt form can be characterized as having a melting point at about 169-176° C.; about 170-174° C., about 171-173° C., about 171-172° C., or about 171° C. The high melt form is distinguishable from at least one other form of 3-fluoro-7-(2,2,2-trifluoroethoxy)phenoxathiin-10,10-dioxide, which melts at about 158-163° C., typically about 160-162° C. The high melt form also can be characterized as containing less than about 1% H₂O, about 1%-0.001% H₂O, about 0.5%-0.01% H₂O, about 0.05%-0.01% H₂O, or about 0.02% H₂O, as determined by the Karl Fischer method. In addition, the high melt form can be characterized as having an attenuated total reflectance Fourier transform infrared spectrum at 1480-1440 cm⁻¹ substantially identical to FIG. 2(a) of the aforementioned application, having an attenuated total reflectance Fourier transform infrared spectrum at 970-800 cm⁻¹ substantially identical to FIG. 2(a) of the aforementioned application, or having an attenuated total reflectance Fourier transform infrared spectrum substantially identical to FIG. 2(a) of the aforementioned application. The attenuated total reflectance Fourier transform infrared spectrum of the high melt form is distinguishable from the attenuated total reflectance Fourier transform infrared spectrum at 970-800 cm⁻¹ and 1480-1440 cm⁻¹ of another form of CX157, which is substantially identical to FIG. 2(b) of the aforementioned application. The high melt form can further be characterized as dissolving at about 75-85° C., about 75-80° C., about 75-78° C., or about 75-77° C. in a solvent that is 10% (v/v) water in acetic acid when the ratio (w/v) of compound to solvent is about 1.6 g:10 mL.

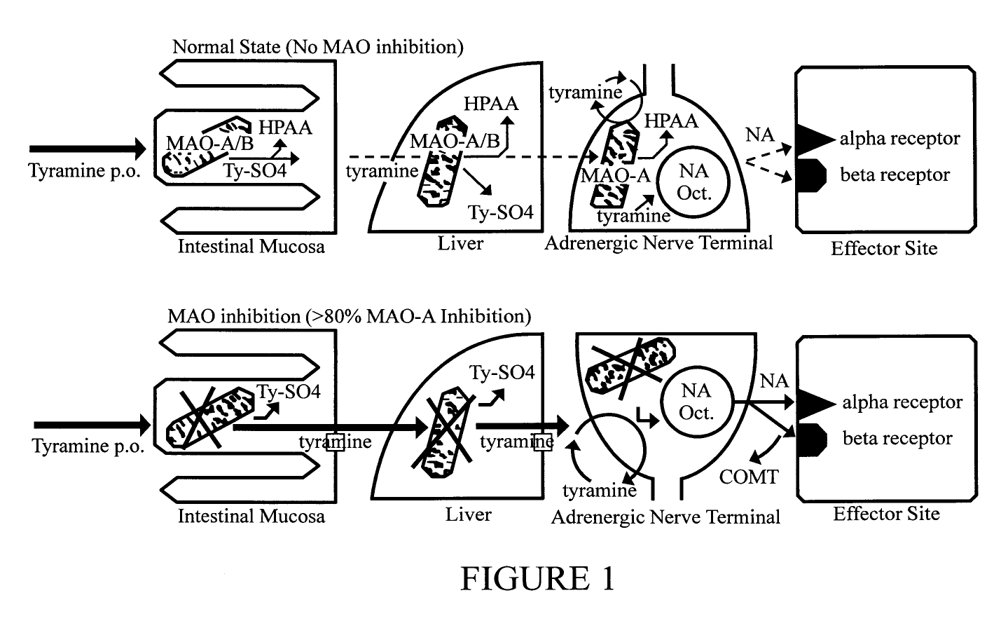

The high melt form can be characterized as having a major x-ray powder diffraction peak at about d spacings 4.0, 4.4 and/or 8.0. The high melt form can be characterized as substantially lacking an x-ray powder diffraction peak at about d spacings 10.3, 7.3, and/or 3.65. The high melt form can be characterized as having a major x-ray powder diffraction peak at about 2θ=11.0°, 20.1°, and/or 22.2°, using CuK_{α} radiation. The high melt form also can be characterized as substantially lacking an x-ray powder diffraction peak at 2θ=8.5°, 12.0°, and/or 24.6°, using CuK_{α} radiation. The high melt form also can be characterized as having an x-ray powder diffraction pattern substantially identical to FIG. 1(a) of the aforementioned application. The x-ray powder diffraction pattern of the high melt form is distinguishable from the x-ray powder diffraction properties of another form of CX157, which has major peaks at about d spacings 10.3, 7.3, and/or 3.65, and about 2θ=11.0°, 20.1°, and/or 22.2°, using CuK_{α} radiation, and has an x-ray powder diffraction pattern substantially identical to FIG. 1(b) of the aforementioned application.

### B. Separating Layer

The separating layer between the active-containing core and the enteric layer is not required, but is a particular feature of the formulation. The functions of the separating layer, if desired, are to provide a smooth base for the application of the enteric layer, to prolong the resistance of the pellet to acid conditions, and/or to improve stability by inhibiting any interaction between the drug and the enteric polymer in the enteric layer.

The smoothing function of the separating layer is purely mechanical, the objective of which is to improve the coverage of the enteric layer and to avoid thin spots in it, caused by bumps and irregularities on the core. Accordingly, the more smooth and free of irregularities the core can be made, the less material is needed in the separating layer, and the need for the smoothing characteristic of the separating layer can be avoided entirely when the active is of extremely fine particle size and the core is made as close as possible to truly spherical.

When a pharmaceutically acceptable non-reducing sugar is added to the separating layer, the pellet's resistance to acid conditions can be markedly increased. Accordingly, such a sugar can be included in the separating layer applied to the cores, either as a powdered mixture, or dissolved as part of the sprayed-on liquid. A sugar-containing separating layer can reduce the quantity of enteric polymer required to obtain a given level of acid resistance. Use of less enteric polymer can reduce both the materials cost and processing time, and also can reduce the amount of polymer available to react with active. The inhibition of any core/enteric layer interaction is mechanical. The separating layer physically keeps the components in the core and enteric layers from coming into direct contact with each other. In some cases, the separating layer can also act as a diffusional barrier to migrating core or enteric layer components dissolved in product moisture. The separating layer can also be used as a light barrier by opacifying it with agents such as titanium dioxide, iron oxides and the like.

In general, the separating layer can include coherent or polymeric materials, and finely powdered solid excipients which constitute fillers. When a sugar is used in the separating layer, it is applied in the form of an aqueous solution and constitutes part of or the whole of the coherent material which sticks the separating layer together. In addition to or instead of the sugar, a polymeric material can also be used in the separating layer. For example, substances such as hydroxypropylmethylcellulose, polyvinylpyrrolidone, hydroxypropylcellulose and the like can be used in small amounts to increase the adherence and coherence of the separating layer.

A filler excipient also can be used in the separating layer to increase the smoothness and solidity of the layer. Substances such as finely powered talc, silicon dioxide and the like are universally accepted as pharmaceutical excipients and can be added as is convenient in the circumstances to fill and smooth the separating layer.

In general, the amount of sugar in the separating layer can be in the range of from about 2% to about 10% of the product, when a sugar is used at all, and the amount of polymeric or other sticky material can be in the range of from about 0.1 to about 5%. The amount of filler, such as talc, can be in the range of from about 5 to about 15%, based on final product weight.

The separating layer can be applied by spraying aqueous solutions of the sugar or polymeric material, and dusting in the filler as has been described in the preparation of an active layer. The smoothness and homogeneity of the separating layer can be improved, however, if the filler is thoroughly dispersed as a suspension in the solution of sugar and or polymeric material, and the suspension is sprayed on the core and dried, using equipment as described above in the preparation of cores with active layers.

### C. Enteric Layer

The enteric layer is comprised of an enteric polymer, which can be chosen for compatibility with the active ingredient. The polymer can be one having only a small number of carboxylic acid groups per unit weight or repeating unit of the polymer. A particular enteric polymer is hydroxypropylmethylcellulose acetate succinate (HPMCAS), which product is defined as containing not less than 4% and not more than 28% of succinoyl groups, which are the only free carboxylic groups in the compound. See Japanese Standards of Pharmaceutical Ingredients 1991, page 1216-21, Standard No. 19026. HPMCAS is available from Shin-Etsu Chemical Co., Ltd., Tokyo, Japan, under the trademark AQOAT. It is available in two particle size grades and three molecular weight ranges. For example, the L grade, having number average molecular weight of 93,000 can be used.

Enteric polymers can be applied as coatings from aqueous suspensions, from solutions in aqueous or organic solvents, or as a powder. One skilled in the art will be able to select from known solvents and/or methods as desired.

The enteric polymer can also be applied according to a method described by Shin-Etsu Chemical Co. Ltd. (Obara, et al., Poster PT6115, AAPS Annual Meeting, Seattle, Wash., Oct. 27-31, 1996). In this method, when the enteric polymer is applied as a powder the enteric polymer is added directly in the solid state to the tablets or pellets while plasticizer is sprayed onto the tablets or pellets simultaneously. The deposit of solid enteric particles is then turned into a film by curing. The curing is done by spraying the coated tablets or pellets with a small amount of water and then heating the tablets or pellets for a short time. This method of enteric coating application can be performed employing the same type of equipment as described above in the preparation of cores with active ingredient layers.

When the enteric polymer is applied as an aqueous suspension, a problem in obtaining a uniform, coherent film often results. In instances in which this problem may arise, a fine particle grade can be used or the particles of polymer can be ground to an extremely small size before application. It is possible either to grind the dry polymer, as in an air-impaction mill or to prepare the suspension and grind the polymer in slurry form. Slurry grinding is generally preferable, particularly since it can be used also to grind the filler portion of the enteric layer in the same step. In some embodiments, it is advisable to reduce the average particle size of the enteric polymer to the range from about 1 micrometer to about 5 micrometers, particularly no larger than 3 micrometers.

When the enteric polymer is applied in the form of a suspension, the suspension is typically maintained homogeneous. Such precautions include maintaining the suspension in a gently stirred condition, but not stirring so vigorously as to create foam, and assuring that the suspension does not stand still in eddies in nozzle bodies, for example, or in over-large delivery tubing. Frequently, polymers in suspension form will agglomerate if the suspension becomes too warm, and the critical temperature can be as low as 30° C. in individual cases. Since spray nozzles and tubing are exposed to hot air in the usual fluid bed type equipment, care must be taken to assure that the suspension is kept moving briskly through the equipment to cool the tubing and nozzle. When HPMCAS is used, in particular, it is advisable to cool the suspension below 20° C. before application, to cool the tubing and nozzle by pumping a little cold water through them before beginning to pump the suspension, and to use supply tubing with as small a diameter as the spray rate will allow so that the suspension can be kept moving rapidly in the tubing.

In one embodiment, one can apply the enteric polymer as an aqueous solution whenever it is possible to do so. In the case of HPMCAS, dissolution of the polymer can be obtained by neutralizing the polymer, particularly with ammonia. Neutralization of the polymer can be obtained merely by adding ammonia, preferably in the form of aqueous ammonium hydroxide to a suspension of the polymer in water; complete neutralization results in complete dissolution of the polymer at about pH 5.7-5.9. Good results are also obtained when the polymer is partially neutralized by adding less than the equivalent amount of ammonia. In such case, the polymer which has not been neutralized remains in suspended form, suspended in a solution of neutralized polymer. The particle size of the polymer can be controlled when such a process is to be used. Use of neutralized polymer more readily provides a smooth, coherent enteric layer than when a suspended polymer is used, and use of partially neutralized polymer provides intermediate degrees of smoothness and coherency. Particularly when the enteric layer is applied over a very smooth separating layer, excellent results can be obtained from partially neutralized enteric polymer.

The extent of neutralization can be varied over a range without adversely affecting results or ease of operation. For example, the extent of neutralization can range from about 25% to about 100% neutralization. Another particular condition is from about 45% to about 100% neutralization, and another condition is from about 65% to about 100%. Still another particular manner of neutralization is from about 25% to about 65% neutralized. It may be found, however, that the enteric polymer in the resulting product, after drying, is neutralized to a lesser extent than when applied. When neutralized or partially neutralized HPMCAS is applied, the HPMCAS in the final product can be from about 0% to about 25% neutralized, more particularly from about 0% to about 15% neutralized.

A plasticizer can be used with enteric polymers for improved results. In the case of HPMCAS, a particular plasticizer can be triethyl citrate, used in an amount up to about 15%-30% of the amount of enteric polymer in aqueous suspension application. When a neutralized HPMCAS is employed, either lower levels or no plasticizer can be required. Minor ingredients, such as antifoam, suspending agents when the polymer is in suspended form, and surfactants to assist in smoothing the film, are also commonly used. For example, silicone anti-foams, surfactants such as polysorbate 80, sodium lauryl sulfate and the like and suspending agents such as carboxymethylcellulose, vegetable gums and the like, can commonly be used at amounts in the general range up to 1% of the product.

Usually, an enteric layer is filled with a powdered excipient such as talc, glyceryl monostearate or hydrated silicon dioxide to build up the thickness of the layer, to strengthen it, to reduce static charge, and to reduce particle cohesion. Amounts of such solids in the range of from about 1% to about 10% of the final product can be added to the enteric polymer mixture, while the amount of enteric polymer itself can be in the range from about 5% to about 25%, more particularly, from about 10% to about 20%.

Application of the enteric layer to the pellets follows the same general procedure previously discussed, using fluid bed type equipment with simultaneous spraying of enteric polymer solution or suspension and warm air drying. Temperature of the drying air and the temperature of the circulating mass of pellets are typically kept in the ranges advised by the manufacturer of the enteric polymer.

### D. Finishing Layer

A finishing layer over the enteric layer is not necessary in every case, but can improve the elegance of the product and its handling, storage and machinability and can provide further benefits as well. The simplest finishing layer is simply a small amount, about less than 1% of an anti-static ingredient such as talc or silicon dioxide, simply dusted on the surface of the pellets. Another simple finishing layer is a small amount, about 1%, of a wax such as beeswax melted onto the circulating mass of pellets to further smooth the pellets, reduce static charge, prevent any tendency for pellets to stick together, and increase the hydrophobicity of the surface.

More complex finishing layers can constitute a final sprayed-on layer of ingredients. For example, a thin layer of polymeric material such as hydroxypropylmethylcellulose, polyvinylpyrrolidone and the like, in an amount such as from about 2% up to about 10%, can be applied. The polymeric material can also carry a suspension of an opacifier, a bulking agent such as talc, or a coloring material, particularly an opaque finely divided color agent such as red or yellow iron oxide. Such a layer quickly dissolves away in the stomach, leaving the enteric layer to protect the active ingredient, but provides an added measure of pharmaceutical elegance and protection from mechanical damage to the product.

Finishing layers to be applied to the present product are of essentially the same types commonly used in pharmaceutical science to smooth, seal and color enteric products, and can be formulated and applied in the usual manners.

The following Examples set out the preparation of a number of different enteric granules consistent with, and that exemplifies the teachings provided herein. The Examples are intended further to enlighten the reader about the present enteric presentations and their methods of manufacture; additional variations within the concept of the invention will be clear to one skilled in the art and their preparation will be within the scientist's competence.

### EXAMPLES

### Example 1

Enteric Capsules of 3-fluoro-7-(2,2,2-trifluoroethoxy)phenoxathiin 10,10-dioxide (60 mg/capsule)

Materials

| Core | | |
|---|---|---|
| Sucrose-starch nonpareils, 30-35 mesh | 134.15 | mg |
| Active layer | | |
| 3-fluoro-7-(2,2,2-trifluoroethoxy)phenoxathiin | 60 | mg |
| 10,10-dioxide | | |
| Sucrose | 25.72 | mg |
| Hydroxypropylmethylcellulose | 12.89 | mg |
| Separating layer | | |
| Hydroxypropylmethylcellulose | 9.45 | mg |
| Sucrose | 28.24 | mg |
| Talc, 500 mesh | 50.21 | mg |
| Enteric layer | | |
| HPMCAS-LF | 65.66 | mg |
| Triethyl citrate | 13.14 | mg |
| Talc, 500 mesh | 39.66 | mg |
| Finishing Layer | | |
| Color mixture white (HPMC + titanium dioxide) | 43.02 | mg |
| HPMC | 10.78 | mg |

| | |
|---|---|
| Talc | Trace |

The active layer is built up by suspending 3-fluoro-7-(2,2,2-trifluoroethoxy)phenoxathiin 10,10-dioxide 25% w/w in a binder solution consisting of 6.4% w/w sucrose and 3.2% w/w hydroxypropylmethylcellulose (HPMC). The resulting suspension is then passed through a Coball Mill (Fryma Mashinen AG, Rheinfelden, Switzerland) Model MS-12 to reduce the particle size of the bulk drug. The milled suspension is applied to 1.5 kg of sucrose starch non-pareils in a fluid bed dryer fitted with a Wurster column. Upon completing the application of the desired quantity of active ingredient suspension, the core pellets are completely dried in the fluid bed dryer.

The separating layer which contains talc 12% w/w, sucrose 6.75% w/w and hydroxypropylmethylcellulose 2.25% w/w is then applied as an aqueous suspension to the active core pellets. Upon completing the application of the desired quantity of suspension, the pellets are completely dried in the fluid bed dryer.

The enteric coating aqueous suspension contains hydroxypropylmethylcellulose acetate succinate type LF 6% w/w, talc 1.8% w/w, triethyl citrate 1.2% w/w which is fully neutralized by the addition of 0.47% w/w ammonium hydroxide. This enteric coating suspension is applied to the separation layer coated pellets. Upon completing the application of the desired quantity of enteric coating suspension, the pellets are completely dried in the fluid bed dryer and a small quantity of talc is added to reduce static charge.

A finishing layer is then applied which contains color mixture white (comprised of titanium dioxide and hydroxypropylmethylcellulose) 8% w/w and hydroxypropylmethylcellulose 2% w/w. Upon completing the application of the desired quantity of color coating suspension, the pellets are completely dried in the fluid bed dryer and a small quantity of talc is added to reduce static charge. The resulting pellets are assayed for active content and filled into capsules to provide 60 mg of 3-fluoro-7-(2,2,2-trifluoroethoxy)phenoxathiin 10,10-dioxide.

### Example 2

### 60 mg CX157/Capsule

Materials

| Beads | |
|---|---|
| Microcrystalline cellulose, | 30.00 mg |
| 32-42 mesh | |
| CX157 layer | |
| CX157 | 60 |
| B-lactose, 5 μm particle size | 41.27 |
| Cross-linked polyvinylpyrrolidone | 6.00 |
| Magnesium stearate | 1.20 |
| Colloidal silicon dioxide | 0.30 |
| Talc | 1.50 |
| Hydroxypropylcellulose | 0.62 |
| Separating layer | |
| Talc | 18.50 |
| Hydroxypropylcellulose | 0.16 |
| Enteric layer | |
| HPMCAS-LF, 3 μm particle size | 34.30 |
| Sorbitan sesquioleate | 0.0002 |
| Triethyl citrate | 6.90 |
| Talc | 10.30 |
| Finishing layer | |
| Titanium dioxide | 8.66 |
| Talc | 4.33 |
| Hydroxypropylmethylcellulose | 3.25 |

The CX157 layer is added in a CF granulator, at a batch size of 5.5 kg. All of the ingredients of the CX157 layer except the CX157, the lactose and the talc are dissolved or suspended in water, and the liquid is slowly sprayed onto the circulating beads and used to adhere the CX157, lactose and talc in building up the CX157 layer.

Similarly, the separating layer is built up in the CF granulator by dissolving the hydroxypropylcellulose in water, and using the solution to adhere the talc on top of the CX157 layer.

The enteric layer is built up in a fluidized bed granulator provided with a top-spray system at a batch size of 1.3 kg. The sesquioleate is dissolved along with the triethyl citrate in water, and the micronized HPMCAS-LF is carefully dispersed and suspended in the cooled solution for spraying into the fluidized bed, maintaining the temperature of the liquid below 15° C. The temperature of the fluidizing air is 70°-80° C. When the HPMCAS-LF suspension and the talc had been completely added, the batch is dried, and the finishing layer is added in the fluidized bed granulator as well. All of the ingredients of the finishing layer are dissolved or suspended in water, and the suspension is sprayed into the batch, maintaining the fluidized air at 70°-80° C.

Finally, the batch is filled into #3 gelatin capsules.

### Example 3

### 60 mg CX-157/Capsule

Materials

| Beads | |
|---|---|
| Sucrose--starch nonpareils, | 50.00 mg |
| 24-32 mesh | |
| CX-157 layer | |
| CX-157 | 60 |
| beta.-lactose | 47.77 |
| Cross-linked polyvinylpyrrolidone | 7.00 |
| Polyvinylpyrrolidone | 0.53 |
| Separating layer | |
| Hydroxypropylcellulose | 7.00 |
| Talc | 14.00 |
| Enteric layer | |
| HPMCAS-LS, Shin-Etsu 3 μm | 31.70 |
| average particle size | |
| Triethyl citrate | 6.60 |
| Talc | 4.70 |
| Titanium dioxide | 4.70 |
| Sodium dodecylbenzenesulfonate | 0.30 |
| Finishing layer | |
| Titanium dioxide | 4.20 |
| beta.-lactose | 4.20 |
| Hydroxypropylmethylcellulose | 2.40 |
| Powder layer | |
| Talc | 0.50 |

The product is made in a CF granulator. The powder layer is applied after the product is dried, in a simple rotating pan without air flow. Each dose of completed granules is filled in #3 gelatin capsules.

### Example 4

### 60 mg CX-157/Capsule

Materials

| | |
|---|---|
| Beads | |
| Sucrose--starch nonpareils | 50.00 mg |
| 24-32 mesh | |
| CX-157 layer | |
| CX-157 | 60 |
| beta.-lactose | 44.77 |
| Cross-linked polyvinylpyrrolidone | 7.00 |
| Polyvinylpyrrolidone | 0.56 |
| Talc | 3.00 |
| Separating layer | |
| Polyvinylpyrrolidone | 2.44 |
| Talc | 18.00 |
| Enteric layer | |
| HPMCAS-LS, 3 μm particle size | 30.70 |
| Triethyl citrate | 6.40 |
| Sodium dodecylbenzenesulfonate | 0.30 |
| Talc | 4.60 |
| Titanium dioxide | 4.60 |
| Finishing layer | |
| Titanium dioxide | 1.0 |
| β-lactose | 3.80 |
| Hydroxypropylmethylcellulose | 3.80 |
| Powder layer Talc | 0.50 |
| | Total Weight - 192.80 mg. |

The product is made in substantially the same manner as Example 3 above.

### Example 5

### 60 mg CX157/Capsule

Materials

| | |
|---|---|
| Beads | |
| Sucrose--starch nonpareils, | 107.66 mg |
| 20-25 mesh | |
| CX-157 layer | |
| CX-157 | 60 |
| Hydroxypropylmethylcellulose | 3.74 |
| Separating layer | |
| Hydroxypropylmethylcellulose | 2.37 |
| Enteric layer | |
| HPMCAS-LF | 23.60 |
| Triethyl citrate | 4.72 |
| Talc 500 | 7.09 |
| mesh | |

The product is made in a CF granulator at a batch size of 1.0 kg. The CX157 layer is built up by spraying into the granulator with inlet air temperature of 80° C. a suspension of the CX157 in a 120 mg/gm aqueous solution of hydroxypropyl-methylcellulose. The suspension is applied to the slowly, keeping the inlet temperature of the fluidizing air at about 80° C. When the CX157 suspension addition is complete, the granules are allowed to air dry.

Then the separating layer is built up by spraying into the granulator an aqueous solution of the hydroxypropylmethylcellulose.

The enteric polymer is neutralized with ammonium hydroxide to dissolve it in water. A sufficient amount of water is used to prepare a 5% w/w solution, and sufficient ammonium hydroxide (28% ammonia solution) is added to achieve a pH of about 6.9. After the polymer had been neutralized, the triethyl citrate and talc are added to the solution, and gently stirred to suspend the talc. Then the suspension is applied to the subcoated granules in the granulator, using an inlet air temperature of about 70° C. After completing the enteric coating application, the pellets are placed onto a paper-lined tray and dried in the dry house at 110° F. for 3 hours. The pellets are then filled into size #3 gelatin capsules.

### Example 6

### 60 mg CX157/Capsule

Materials

| | |
|---|---|
| Beads | |
| Sucrose--starch nonpareils, | 99.76 mg |
| 20-25 mesh | |
| CX-157 layer | |
| CX-157 | 60 |
| Hydroxypropylmethylcellulose | 4.50 |
| Separating layer | |
| Hydroxypropylmethylcellulose | 3.30 |
| Talc, 500 mesh | 7.60 |
| Enteric layer | |
| HPMCAS-LF | 16.11 |
| Triethyl citrate | 3.22 |
| Talc, 500 mesh | 12.26 |
| Finishing Layer | |
| Talc | Trace |

The product is made in the same manner used in Example 5, except that the CX157 suspension is passed through a Tri-Homo Disperser—Homogenizer (Tri-Homo Corporation, Salem, Mass., U.S.A.) mill. In order to alleviate static charge and to improve flow, a small amount of talc is added to the pellets prior to capsule filling.

### Example 7

### 60 mg CX157/Capsule

Materials

| Beads | |
|---|---|
| Sucrose--starch nonpareils | 109.86 mg |
| 20-25 mesh | |
| CX-157 layer | |
| CX-157 | 60 |
| Hydroxypropylmethylcellulose | 4.48 |
| Separating layer | |
| Hydroxypropylmethylcellulose | 4.51 |
| Enteric layer | |
| HPMCAS-LS | 24.34 |
| Talc, 500 mesh | 2.44 |
| Triethyl citrate | 7.31 |
| Polysorbate 80 | 0.25 |
| Emulsion silicone solids | 0.10 |
| Carboxymethylcellulose | 0.18 |
| Finishing layer | |
| Hydroxypropylmethylcellulose | 8.34 |
| Titanium dioxide | 2.78 |
| Propylene glycol | 3.70 |

The CX157 layer is built up by suspending CX157 in a 4% w/w solution of the hydroxypropylmethylcellulose in water, and milling the suspension with a CoBall Mill (Fryma Mashinen AG, Rheinfelden, Switzerland) model MS-12. A fluid bed dryer with a Wurster column is used to make this product at a batch size of 1.0 kg. The separating layer is added from a 4% w/w solution of the hydroxypropylmethylcellulose in water.

In order to prepare the enteric coating suspension, purified water is cooled to 10° C. and the polysorbate, triethyl citrate and silicone emulsion are added and dispersed or dissolved. Then the HPMCAS and talc are added and agitated until homogeneity is obtained. To this suspension, a carboxymethylcellulose aqueous solution, 0.5% w/w, is added and blended thoroughly. The enteric suspension is maintained at 20° C. during the coating process. The enteric suspension is then added to the partially completed pellets in the Wurster column at a spray rate of about 15 ml/min, holding the temperature of the inlet air at about 50° C. The product is dried in the Wurster at 50° C. when the enteric suspension had been fully added, and then dried on trays for 3 hours in a dry house at 60° C. A finishing layer is then applied which consisted of a 4.5% w/w/hydroxypropylmethylcellulose solution containing titanium dioxide and propylene glycol as plasticizer. The pellets are completely dried in the fluid bed dryer and then are then filled in size 3 gelatin capsules.

### Example 8

### 60 mg CX157/Capsule

Materials

| | |
|---|---|
| Beads | |
| Sucrose--starch nonpareils | 59.43 mg. |
| 20-25 mesh | |
| CX-157 layer | |
| CX-157 | 60 |
| Hydroxypropylmethylcellulose | 4.50 |
| Emulsion silicone solids | 0.04 |
| Separating layer | |
| Hydroxypropylmethylcellulose | 2.26 |
| Talc, 500 mesh | 4.53 |
| Enteric layer | |
| HPMCAS-LS | 18.49 |
| Talc, 500 mesh | 1.85 |
| Triethyl citrate | 5.55 |
| Polysorbate 80 | 0.19 |
| Emulsion silicone solids | 0.07 |
| Finishing layer | |
| Hydroxypropylmethylcellulose | 5.47 |
| Titanium dioxide | 1.82 |
| Propylene glycol | 2.43 |
| Talc | Trace |

The product is made in essentially the same manner as that of Example 7 above, with the exception that approximately 25% of the enteric polymer had been neutralized with ammonium hydroxide prior to addition to the remaining components of the enteric coating suspension.

### Example 9

### 60 mg CX157/Capsule

Materials

| | |
|---|---|
| Beads | |
| Sucrose--starch nonpareils, | 60.33 mg |
| 20-25 mesh | |
| CX-157 layer | |
| CX-157 | 60 |
| Hydroxypropylmethylcellulose | 3.75 |
| Separating layer | |
| Hydroxypropylmethylcellulose | 4.15 |
| Talc, 500 mesh | 12.46 |
| Enteric layer | |
| HPMCAS-LF | 24.82 |
| Triethyl citrate | 4.95 |
| Talc, 500 mesh | 7.45 |
| Finishing Layer | |
| Hydroxypropylmethylcellulose | 8.36 |
| Titanium dioxide | 2.79 |
| Talc | Trace |

The product is made essentially as is the product of Example 7 except that in this instance the HPMCAS-LF is fully neutralized to a pH of 5.7 and complete solubility in water.

### Example 10

### 60 mg CX157/Capsule

Materials

| | |
|---|---|
| Beads | |
| Sucrose - starch nonpareils, | 60.28 mg |
| 20-25 mesh | |
| CX-157 layer | |
| CX-157 | 60 |
| Hydroxypropylmethylcellulose | 3.74 |
| Separating layer | |
| Hydroxypropylmethylcellulose | 2.51 |
| Sucrose | 5.00 |
| Talc, 500 mesh | 10.03 |
| Enteric layer | |
| HPMCAS-LF | 25.05 |
| Triethyl citrate | 5.00 |
| Talc, 500 mesh | 7.52 |
| Finishing layer | |
| Hydroxypropylmethylcellulose | 8.44 |
| Titanium dioxide | 2.81 |
| Talc | Trace |
| | Total Weight - 141.60 mg |

The product is made substantially according to the process used in Example 7. In this instance, the sucrose is dissolved in the water used to form the separating layer, and the HPMCAS-LF is fully neutralized.

### Example 11

60 mg CX157 base/Capsule

Materials

| | |
|---|---|
| Beads | |
| Sucrose--starch nonpareils, | 84.92 mg |
| 20-25 mesh | |
| CX-157 layer | |
| CX-157 | 60 |
| Hydroxypropylmethylcellulose | 4.27 |
| Separating layer | |
| Hydroxypropylmethylcellulose | 2.22 |
| Sucrose | 6.68 |
| Talc, 500 mesh | 11.87 |
| Enteric layer | |
| HPMCAS-LF | 27.36 |
| Triethyl citrate | 5.47 |
| Talc, 500 mesh | 8.22 |
| Finishing layer | |
| Hydroxypropylmethylcellulose | 9.82 |
| Titanium dioxide | 2.55 |
| Yellow iron oxide | 0.72 |
| Talc | Trace |

The product is made substantially according to the process used in Example 10.

Pellets made according to the above examples, and gelatin capsules filled with various batches of such pellets, are thoroughly tested in the manners usual in pharmaceutical science. Results of stability tests show that the pellets and capsules have sufficient storage stability to be distributed, marketed and used in the conventional pharmaceutical manner.

Testing further shows that the pellets and capsules pass the conventional tests for enteric protection under conditions prevailing in the stomach. It has also been shown that the pellets release their load of CX157 acceptably quickly when exposed to conditions prevailing in the small intestine. Accordingly, the present invention is demonstrated to solve the problems which previously are encountered in the formulation of other CX157 pellets.

### Example 12

Enteric Coated Tablets of 3-fluoro-7-(2,2,2-trifluoroethoxy)phenoxathiin 10,10-dioxide (50 mg/Tablet):

TABLE 1

| Material | mg/tablet |
|---|---|
| Mannitol | 37.56 |
| Aerosil | 0.6 |
| CX-157 | 50 |
| Starch NF | 42.84 |
| Starch 1500 | 10 |
| Eudragit L30-D55 | 50 |
| Triethyl citrate | 5 |
| Talc | 2 |
| Stearic Acid | 2 |

All excipients except for Eudragit L-30 D-55 (methacrylic acid-ethyl acrylate copolymer (1:1) dispersion 30 percent) and triethyl citrate are mixed and granulated with water and compressed into tablets. Triethyl citrate and water are homogenized, and Eudragit is added to the homogenized mix to obtain a dispersion that contains about 54% water. The tablets are sprayed with the dispersion in a Glatt Coater (Glatt Muschinen & Apparatebau AG, Pratteln Switzerland) coating pan. The inlet air temperature is 55° C., the outlet air temperature is between 40-44° C., and the spraying rate is 20 rpm. The pan speed is set to 5 rpm.

The tablet dissolution profile is analyzed using United States Pharmacopeia method <724> for coated tablets. After 120 minutes in 0.1N HCl, the tablets are transferred to phosphate buffer solution.

### Example 13

Capsules Containing Enteric Coated Particles of 3-fluoro-7-(2,2,2-trifluoroethoxy)phenoxathiin 10,10-dioxide (50 mg/tablet)

Particles (sugar spheres) for capsule filling are made using the ingredients listed in the following Table:

| Ingredients | mg/capsule |
|---|---|
| Sucrose/Corn Starch Spheres (92:8) | 121 |
| (Suglets ®. NP Pharm Bazainville, France) | |
| CX-157 | 50 |
| Polyethylene Glycol (PEG 6000 NF) | 2.0 |
| Hydroxypropylmethylcellulose (Pharmacoat 606 Shin-Etsu) | 8.0 |

PEG 6000 is mixed with water to form a solution. CX-157 is then added and the solution is mixed. Hydroxypropylmethylcellulose is added to water, and the two solutions are combined and mixed. Suglets are placed in a Wurster fluid bed drier and the combined solution is sprayed on to the Suglets. The inlet temperature is 55° C., and the outlet temperature is between 29° C. and 47° C. The spray rate is between 8 and 16 gram/min. The airflow rate is between 50-120 m³/hour.

The particles (sugar spheres) are then coated with an enteric coating, as described below:

| Ingredient | |
|---|---|
| Eudragit L-30 D-55 (mg/capsule) | 54 |
| Triethyl citrate (mg/capsule) | 5.4 |
| % coating | ~30 |

The percentage of coating is calculated as Eudragit weight/CX-157 coated particle weight.

Triethyl citrate and water are homogenized, and Eudragit is added to attain a dispersion which contains 45.4% water. The drug coated pellets are placed in the Wurster fluid bed drier a second time. The dispersion is sprayed at a rate of between 8 and 16g/min. The inlet temperature is between 33° C. and 48° C., and the outlet temperature is between 25° C. and 45° C. The airflow rate is between 40 and 120 m³/hour. After coating, the enteric coated pellets are dried for 90 minutes. Six batches of enteric coated pellets are formed with different amounts of coating in each batch.

The enteric coated particles are then filled into HDP #1 capsules. The dissolution profile of the capsules batches in HCl 0.1 N, based on USP procedures, is taken. The dissolution profile of the capsules in phosphate buffer is measured.

The dissolution profile would show that the enteric coating is effective in protecting the spheres from being dissolved in the stomach, thereby eliminating cheese effect in subjects who are treated with the capsules. Capsules comprising spheres as in such capsules would be effective in treating depression because the spheres maintain integrity in stomach-like conditions, and are easily soluble in intestine-like conditions.

The above examples also can be used to formulate tables and capsules in quantities of active ingredient ranging from, for example, 50-500 mg, including 100-200 mg.

Since modifications will be apparent to those of skill in this art, it is intended that this invention be limited only by the scope of the appended claims.

1. An enteric oral pharmaceutical product comprising a phenoxathiin-based MAO-A inhibitor of the following formula: 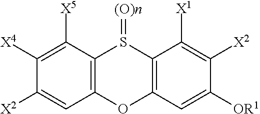
wherein n is 0, 1 or 2; R¹ is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl.

2. The enteric product of claim 1, wherein the phenoxathiin-based MAO-A inhibitor is 3-fluoro-7-(2,2,2-trifluoroethoxy)phenoxathiin 10,10-dioxide.

3. The enteric product of claim 1, wherein said product is a tablet.

4. The enteric product of claim 1, wherein said product is a capsule or a core sheathed in an annular body.

5. The enteric product of claim 1, wherein said product comprises an enteric coating which is essentially not dissolvable in the stomach surrounding a core which comprises said active ingredient.

6. The enteric product of claim 1, wherein said product contains 3-fluoro-7-(2,2,2-trifluoroethoxy)phenoxathiin 10,10-dioxide as the sole active ingredient.

7. The enteric product of claim 2, wherein said 3-fluoro-7-(2,2,2-trifluoroethoxy)phenoxathiin 10,10-dioxide is characterized as having a melting point at about 169-175° C.

8. The enteric product of claim 2 wherein said 3-fluoro-7-(2,2,2-trifluoroethoxy)phenoxathiin 10,10-dioxide is characterized as being in crystalline form and having an x-ray powder diffraction peak at 2θ=11.0°, using CuK_{α} radiation.

9. The enteric product of claim 1, wherein said product comprises:
(a) a core consisting of 3-fluoro-7-(2,2,2-trifluoroethoxy)phenoxathiin 10,10-dioxide and one or more pharmaceutical excipients;
(b) an optional separating layer;
(c) an enteric layer comprising hydroxypropylmethylcellulose acetate succinate (HPMCAS) and a pharmaceutically acceptable excipient; and
(d) an optional finishing layer.

10. The enteric product of claim 9, wherein the separating layer (b) is present.

11. The enteric product of claim 9, wherein the core comprises an inert bead on which the 3-fluoro-7-(2,2,2-trifluoroethoxy)phenoxathiin 10,10-dioxide is deposited as a layer comprising said one or more pharmaceutical excipients.

12. The enteric product of claim 1, wherein said product is a tablet containing about 50 to 500 milligrams of 3-fluoro-7-(2,2,2-trifluoroethoxy)phenoxathiin 10,10-dioxide.

13. An oral pharmaceutical dosage form comprising 3-fluoro-7-(2,2,2-trifluoroethoxy)phenoxathiin 10,10-dioxide and adapted to retard or inhibit the release of 3-fluoro-7-(2,2,2-trifluoroethoxy)phenoxathiin 10,10-dioxide in the stomach.

14. The oral pharmaceutical dosage form of claim 13 that is a tablet, a capsule, or a core sheathed in an annular body.

15. The pharmaceutical dosage form of claim 14 that is a tablet.

16. The pharmaceutical dosage form of claim 14 that is a capsule.

17. The pharmaceutical dosage form of claim 13 comprising an enteric coating.
